(19) United States
(12) Patent Application Publication
(10) Pub. No.: US 2025/0170285 A1
(43) Pub. Date: May 29, 2025

(54) RADIOLABELLED OLIGONUCLEOTIDES AND PROCESS FOR THEIR PREPARATION

(71) Applicant: Hoffmann-La Roche Inc., Little Falls, NJ (US)

(72) Inventor: MARTIN ROBERT EDELMANN, Basel (CH)

(73) Assignee: Hoffmann-La Roche Inc., Little Falls, NJ (US)

(21) Appl. No.: 19/046,794

(22) Filed: Feb. 6, 2025

Related U.S. Application Data
(62) Division of application No. 16/936,980, filed on Jul. 23, 2020.
Application No. 16/936,980 is a continuation of application No. PCT/EP2019/051682, filed on Jan. 24, 2019.

(30) Foreign Application Priority Data
Jan. 26, 2018 (EP) 18153591.5

(51) Int. Cl. A61K 51/04; C07B 59/00; C07H 15/12; C07H 21/00

(52) U.S. Cl. CPC A61K 51/0491; C07B 59/005; C07H 15/12; C07H 21/00

(57) ABSTRACT

The invention comprises radiolabeled oligonucleotide of the formula I

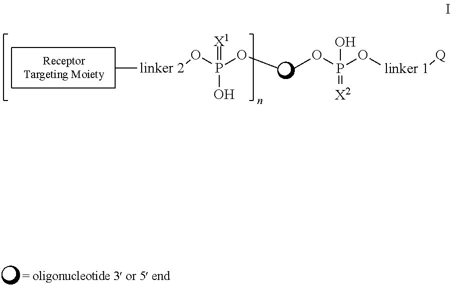

wherein n, X¹, X², the linker 1, the linker 2, Q and the receptor targeting moiety are as defined I the description. The radiolabeled oligonucleotides of the formula I can be used for the determination of the biodistribution and pharmacokinetics of the oligonucleotide in the tissue or body fluid.

### CROSS REFERENCE TO RELATED APPLICATIONS

This application is a Divisional of U.S. application Ser. No. 16/936,980, filed Jul. 23, 2020 which is a continuation of International Application No. PCT/EP2019/051682 having an international filing date of Jan. 24, 2019, the entire contents of which are incorporated herein by reference, and which claims benefit under 35 U.S.C. § 119 to European Patent Application No. 18153591.5 filed on Jan. 26, 2018.

### SEQUENCE LISTING

This application contains a Sequence Listing which has been submitted electronically in XML format and is hereby incorporated by reference in its entirety. Said XML copy, created on Feb. 4, 2025, is named P34626-US-1.xml and is 6,088 bytes in size.

### FIELD OF THE INVENTION

The invention relates to novel radiolabeled oligonucleotide of the formula I

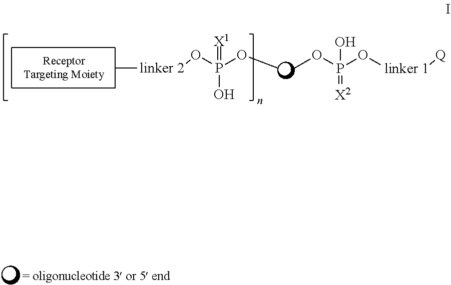

wherein,
n, X¹ and X², the linkers 1 and 2, Q and the receptor targeting moiety are discussed hereinafter, a process for their preparation and to their use for the determination of the biodistribution and pharmacokinetics of the oligonucleotide in the tissue or body fluid.

### SEQUENCE LISTING

The instant application contains a Sequence Listing submitted via EFS-Web and hereby incorporated by reference in its entirety. Said ASCII copy, created on Jul. 21, 2020, is named P34626US_SeqList.txt, and is 1,117 bytes in size.

### BACKGROUND

For an antisense therapeutic approach to be effective, oligonucleotides must be introduced into a patient and must reach the specific tissues to be treated. The biodistribution and pharmacokinetics of a therapeutic drug must be determined as a step preliminary to treatment with the drug. Consequently, there is a need to be able to detect oligonucleotides in body fluids or tissues. Agrawal et al., Clin. Pharmacokinetics 28, 7 (1995), reviews certain aspects of the pharmacokinetics of antisense oligonucleotides. Another well-established approach used in in vivo pharmacokinetic studies of pharmacological compounds such as antisense oligonucleotides entails radiolabeling the compounds to enable detection. In animal models, radiolabeled oligonucleotides have been administered to the animal and their distribution within body fluids and tissues has been assessed by extraction of the oligonucleotides followed by autoradiography (See Agrawal et al., Proc. Natl. Acad. Sci. 88, 7595-7599 (1991).

³⁵S-labeling is an established and wide-spread technique. For biological studies, ³⁵S-labeled oligonucleotide phosphorothioates have been prepared using H-phosphonate chemistry (See Garegg et al., Chem. Scr. 25, 280-282 (1985).

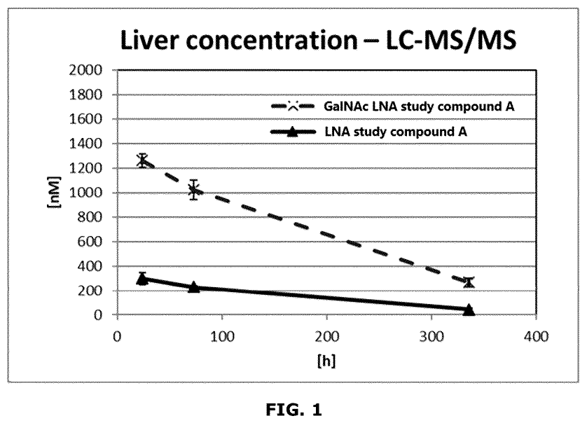

Radioisotopic labeling of synthetic oligonucleotides with ¹⁴C and ³H is currently accomplished by using the well-established solid-phase automated synthesis. In this approach, the assembly of ¹⁴C or ³H nucleoside phosphoramidite requires a two-step process as shown in FIG. 1 of U.S. Pat. No. 5,847,104. However, several disadvantages are associated with this method. Since the radioisotope is introduced in the very first step, (a) the radiochemical yield after two steps is limited; (b) this operation often suffers a dilution problem, namely, the natural abundance isotope is usually blended in as a carrier in order to maintain a manageable synthetic scale, resulting in lower specific activity of the final oligos and (c) the phosphoramidite 3 (FIG. 1) is a reactive species prone to degradation which as the final radioactive precursor leads to stringent storage and transportation requirements.

In view of the deficiencies of the prior art methods other approaches for obtaining radiolabeled oligonucleotides with high specific activity are desirable.

### BRIEF SUMMARY

Object of the invention therefore is to provide a new approach for the radiolabeling of oligonucleotides.

It was found that the objective could be fulfilled with the newly developed radiolabeled oligonucleotide of the formula I

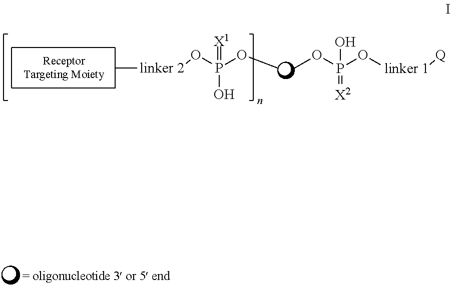

wherein,
n is 0 or 1;
X¹ and X² independently of each other are S or O;
linker 1 is a C₂₋₁₂-alkylene bridge, an ethylene glycol bridge containing 1 to 10 ethylene glycol units or a glycerol based bridge of the formula

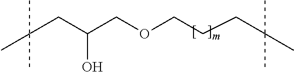

wherein m is an integer of 1 to 6;
linker 2 is an optionally amino group protected amino C₂₋₁₂-alkylene bridge, an amino ethylene glycol bridge containing 1 to 10 ethylene glycol units;
Q stands for a residue of the formula 2a or 2b

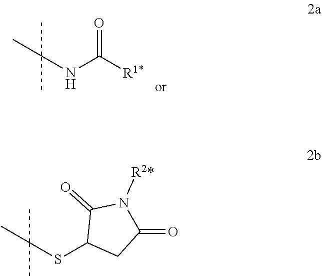

wherein R^{1*} and R^{2*} are radiolabeled C₁₋₆-alkyl groups;
and the receptor targeting moiety is a moiety which adds additional functionality to the oligonucleotide.

### BRIEF DESCRIPTION OF THE FIGURES

The Figures have the following meaning:

In FIG. 1 the liver concentration of a GalNAc study compound A (dotted line) and a study compound A without GalNAc (continuous line) have been compared with LC-MS/MS.

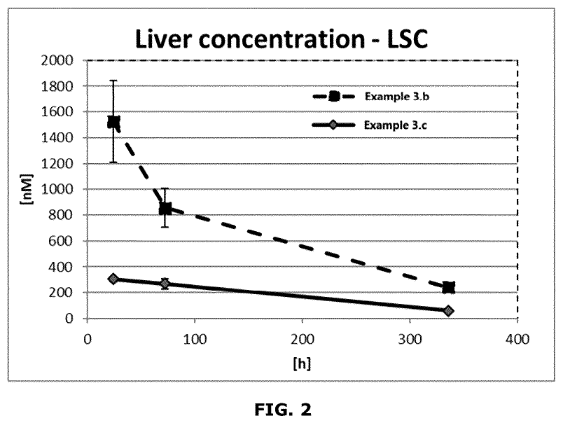

In FIG. 2 the liver concentration of the tritium labeled compounds of Example 3b (dotted line) and Example 3c (continuous line) have been compared with LSC.

### DETAILED DESCRIPTION

The following definitions are set forth to illustrate and define the meaning and scope of the various terms used to describe the invention herein.

The term “C₁₋₆-alkyl” denotes a monovalent linear or branched saturated hydrocarbon group of 1 to 6 carbon atoms, and in more particular embodiments 1 to 4 carbon atoms. Examples of C₁₋₆-alkyl include methyl, ethyl, propyl, isopropyl, n-butyl, i-butyl, sec-butyl, or t-butyl, preferably methyl or ethyl, more preferably ethyl.

The term “C₂₋₁₂-alkyl” likewise denotes a monovalent linear or branched saturated hydrocarbon group of 2 to 12 carbon atoms, in a more particular embodiment 4 to 8 carbon atoms and even more particular embodiment of 6 carbon atoms. Particular examples are butyl, pentyl, hexyl, heptyl or octyl and its isomers, but preferably n-hexyl.

The term “C₂₋₁₂-alkylene bridge” stands for a bivalent linear or branched saturated hydrocarbon group of 2 to 12 carbon atoms, in a more particular embodiment 4 to 8 carbon atoms and in an even more particular embodiment of 6 carbon atoms. Particular examples are butylene, pentylene, hexylene, heptylene or octylene and its isomers, but preferably n-hexylene.

The term “amino C₂₋₁₂-alkylene bridge” stands for a bivalent group comprising an amino group attached to a branched saturated hydrocarbon group of 2 to 12 carbon atoms, in a more particular embodiment 4 to 8 carbon atoms and in an even more particular embodiment of 6 carbon atoms. Particular examples are amino butylene, amino pentylene, amino hexylene, amino heptylene or amino octylene and its isomers, but preferably amino n-hexylene (—NH—(CH₂)₆—).

The term “ethylene glycol units” stands for units of the formula —(CH₂)₂—O— which as a bridging unit can contain 1 to 10 ethylene glycol units, preferably 2 to 6 ethylene glycol units.

The term “glycerol unit glycerol based bridge” is characterized by the formula

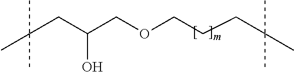

wherein m is an integer of 1 to 6, preferably 1 to 3, more preferably 1.

The term “amino-protecting group” denotes groups intended to protect an amino group and includes benzoyl, benzyloxycarbonyl, carbobenzyloxy (CBZ or Z), 9-fluorenylmethyloxycarbonyl (FMOC), p-methoxybenzyloxycarbonyl, p-nitrobenzyloxycarbonyl, t-butoxycarbonyl (BOC), and trifluoroacetyl. Further examples of these groups are found in T. W. Greene and P. G. M. Wuts, “Protective Groups in Organic Synthesis”, 2nd ed., John Wiley & Sons, Inc., New York, NY, 1991, chapter 7; E. Haslam, “Protective Groups in Organic Chemistry”, J. G. W. McOmie, Ed., Plenum Press, New York, NY, 1973, Chapter 5, and T. W. Greene, “Protective Groups in Organic Synthesis”, John Wiley and Sons, New York, NY, 1981.

The term oligonucleotide as used herein is defined as it is generally understood by the skilled person as a molecule comprising two or more covalently linked nucleotides. For use as a therapeutically valuable oligonucleotide, oligonucleotides are typically synthesized as 7-30 nucleotides in length.

The oligonucleotides may consist of optionally modified DNA, RNA or LNA nucleoside monomers or combinations thereof.

The LNA nucleoside monomers are modified nucleosides which comprise a linker group (referred to as a biradicle or a bridge) between C2′ and C4′ of the ribose sugar ring of a nucleotide. These nucleosides are also termed bridged nucleic acid or bicyclic nucleic acid (BNA) in the literature.

Optionally modified as used herein refers to nucleosides modified as compared to the equivalent DNA, RNA or LNA nucleoside by the introduction of one or more modifications of the sugar moiety or the nucleo base moiety. In a preferred embodiment the modified nucleoside comprises a modified sugar moiety, and may for example comprise one or more 2′ substituted nucleosides and/or one or more LNA nucleosides. The term modified nucleoside may also be used herein interchangeably with the term “nucleoside analogue” or modified “units” or modified “monomers”.

The DNA, RNA or LNA nucleosides are as a rule linked by a phosphodiester (P═O) and/or a phosphorothioate (P═S) internucleoside linkage which covalently couples two nucleosides together.

Accordingly, in some oligonucleotides all internucleoside linkages may consist of a phosphodiester (P═O), in other oligonucleotides all internucleoside linkages may consist of a phosphorothioate (P═S) or in still other oligonucleotides the sequence of internucleoside linkages vary and comprise both phosphodiester (P═O) and phosphorothioate (P═S) internucleoside.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are described with capital letters A, T, G and ^{Me}C (5-methyl cytosine) for LNA nucleoside and with small letters a,t,g,c and ^{Me}c for DNA nucleosides. Modified nucleobases include but are not limited to nucleobases carrying protecting groups such as t-butylphenoxyacetyl, phenoxyacetyl, benzoyl, acetyl, i-butyryl or dimethylformamidino (see Wikipedia, Phosphoramidit-Synthese, of Mar. 24, 2016).

Preferably the oligonucleotide consists of optionally modified DNA or LNA nucleoside monomers or combinations thereof and is 10 to 25 nucleotides in length.

The principles of the oligonucleotide synthesis are well known in the art und well described in literature and public for a like Wikipedia (see e.g. Oligonucleotide synthesis; Wikipedia, the free encyclopedia; of Mar. 15, 2016).

Larger scale oligonucleotide synthesis nowadays is carried automatically using computer controlled synthesizers.

As a rule, oligonucleotide synthesis is a solid-phase synthesis, wherein the oligonucleotide being assembled is covalently bound, via its 3′-terminal hydroxy group, to a solid support material and remains attached to it over the entire course of the chain assembly. Suitable supports are the commercial available macroporous polystyrene supports like the Primer support 5G from GE Healthcare or the NittoPhase®HL support from Kinovate.

The oligonucleotide synthesis in principle is a stepwise addition of nucleotide residues to the 5′-terminus of the growing chain until the desired sequence is assembled.

As a rule, each addition is referred to as a synthetic cycle and in principle consists of the chemical reactions
a₁) de-blocking the protected hydroxyl group on the solid support,
a₂) coupling the first nucleoside as activated phosphoramidite with the free hydroxyl group on the solid support,
a₃) oxidizing or sulfurizing the respective P-linked nucleoside to form the respective phosphotriester (P═O) or the respective phosphorothioate (P═S);
a₄) optionally, capping any unreacted hydroxyl groups on the solid support;
a₅) de-blocking the 5′ hydroxyl group of the first nucleoside attached to the solid support;
a₆) coupling the second nucleoside as activated phosphoramidite to form the respective P-linked dimer;
a₇) oxidizing or sulfurizing the respective P-linked dinucleoside to form the respective phosphotriester (P═O) or the respective phosphorothioate (P═S);
a₈) optionally, capping any unreacted 5′ hydroxyl groups;
a₉) repeating the previous steps as to as until the desired sequence is assembled.

The term “radiolabeled” in the context of the present invention is used for the substituents R^{1*} and R^{2*} which are radiolabeled C₁₋₆-alkyl groups, preferably a radiolabeled C₁₋₄-alkyl groups, more preferably a methyl or ethyl group. A suitable radiolabeling for these groups therefore means the replacement of the natural atoms by its corresponding radioactive isotopes ¹⁴C or ³H, but preferably with ³H.

The term “receptor targeting moiety” stands for a moiety which adds additional functionality to the oligonucleotide.

Such moieties can be selected from any protein receptor target moiety which has the potential to enhance functionality to the oligonucleotide. They include, but are not limited to antibodies or functional peptides or oligonucleotides which target specific molecules like aptamers or non-nucleotide protein receptor target moieties which have the potential to enhance delivery of the oligonucleotide to body tissue or body fluid.

In a preferred embodiment the receptor targeting moiety is an asialglycoprotein receptor targeting moiety, more preferably a GalNAc moiety.

The GalNAc moiety has the formula VII

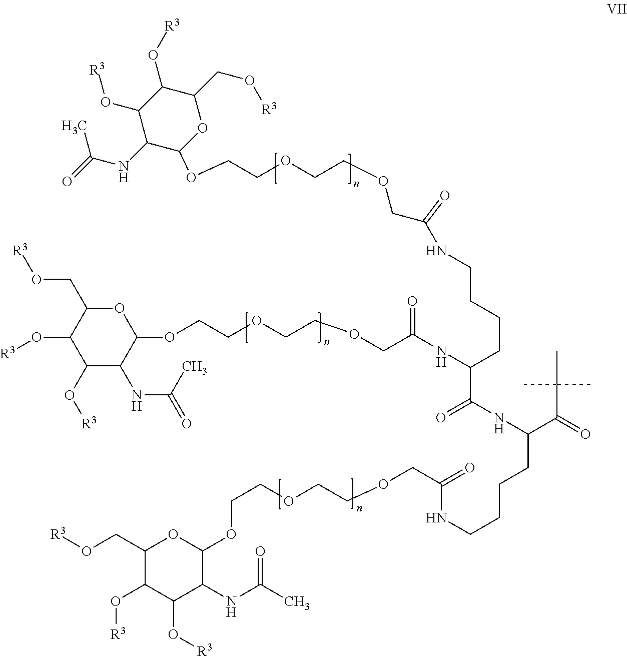

wherein R³ is hydrogen or a hydroxy protecting group and n is an integer from 0 to 10, preferably from 0 to 5, more preferably from 1 to 3, but most preferred is 2, corresponding salts, enantiomers and/or a stereoisomer thereof.

Suitable hydroxy protecting groups are acyl, particularly the C₁₋₁₂-alkylcarbonyl group, more particularly the C₁₋₆-alkylcarbonyl group which is optionally substituted by C₁₋₆-alkyl or phenyl. More preferred is acetyl, pivaloyl or benzoyl, whereby acetyl is the most preferred hydroxy protecting group.

In a preferred embodiment the GalNAc moiety has the formula VII wherein R³ is hydrogen and n is 2.

The GalNAc moiety is connected with linker 2 via a peptide bond —CO—NH—.

The GalNAc cluster compounds can be prepared according to the PCT Publication WO2017021385.

In a preferred embodiment the radiolabeled oligonucleotide of formula 1 Q has the formula 2b and the conjugation is at the 3′ or 5′ end of the oligonucleotide.

In another preferred embodiment radiolabeled oligonucleotide of claim 1 or 2, wherein Q has the formula 2a and the conjugation is at the 3′ or 5′ end of the oligonucleotide.

Particularly preferred rare radiolabeled oligonucleotides of formula 1 wherein Q has the formula 2b and the conjugation is at the 3′ or 5′ end of the oligonucleotide.

In another embodiment the radiolabeled oligonucleotide has the formula Ib

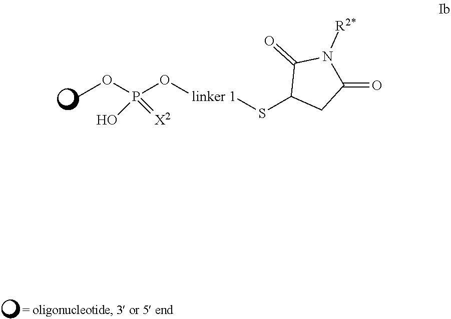

wherein
R^{2*} is radiolabeled C₁₋₆-alkyl
X² is S or O;
linker 1 is a C₂₋₁₂-alkylene bridge, an ethylene glycol bridge containing 1 to 10 ethylene glycol units or a glycerol based bridge of the formula

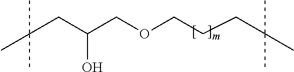

wherein m is an integer of 1 to 6.

In a preferred embodiment the radiolabeled oligonucleotide of the formula Ib has a conjugation at the 3′ end.

In another preferred embodiment of the radiolabeled oligonucleotide of the formula Ib R^{2*} is methyl or ethyl, more preferably ethyl.

In another preferred embodiment of the radiolabeled oligonucleotide of the formula Ib X² is S.

In another preferred embodiment of the radiolabeled oligonucleotide of the formula Ib the linker 1 is a C₂₋₁₂-alkylene bridge, preferably a C₆-alkylene bridge.

Even more preferred is the radiolabeled oligonucleotide of the formula Ib, wherein R^{2*} is methyl or ethyl, preferably ethyl; X² is S and the linker 1 is a C₆-alkylene bridge.

In another embodiment the radiolabeled oligonucleotide has the formula Ic

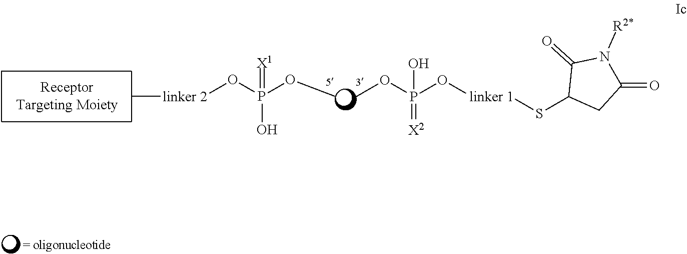

wherein,
R^{2*} is radiolabeled C₁₋₆-alkyl;
X¹ and X² independently of each other are S or O;
linker 1 is a C₂₋₁₂-alkylene bridge, an ethylene glycol bridge containing 1 to 10 ethylene glycol units or a glycerol based bridge of the formula

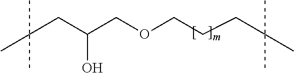

wherein m is an integer of 1 to 6;
linker 2 is an optionally amino group protected amino C₂₋₁₂-alkylene bridge, an amino ethylene glycol bridge containing 1 to 10 ethylene glycol units;
and the receptor targeting moiety is a moiety which adds additional functionality to the oligonucleotide.

The receptor targeting moiety is as defined above, but preferably an asialglycoprotein receptor targeting moiety, more preferably a GalNAc moiety.

In a preferred embodiment of the radiolabeled oligonucleotide of the formula Ic, R^{2*} is methyl or ethyl, more preferably ethyl.

In another preferred embodiment of the radiolabeled oligonucleotide of the formula Ic, X¹ is O and X² is S.

In another preferred embodiment of the radiolabeled oligonucleotide of the formula Ic the linker 1 is a C₂₋₁₂-alkylene bridge, preferably a C₆-alkylene bridge.

In another preferred embodiment of the radiolabeled oligonucleotide of the formula Ic the linker 2 is an amino C₂₋₁₂-alkylene bridge, preferably an amino C₆-alkylene bridge.

In another preferred embodiment of the radiolabeled oligonucleotide of the formula Ic the receptor targeting moiety is a GalNAc moiety of formula V

Even more preferred is the radiolabeled oligonucleotide of the formula Ic, wherein R^{2*} is methyl or ethyl, preferably ethyl; X¹ is O and X² is S; the linker 1 is a C₆-alkylene bridge; the linker 2 is an amino C₆-alkylene bridge and the receptor targeting moiety is a GalNAc moiety of formula V with R³ hydrogen and n=2.

In another embodiment the radiolabeled oligonucleotide has the formulae Id

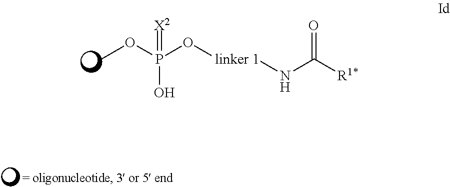

wherein
R^{1*} is radiolabeled C₁₋₆-alkyl
X² is S or O;
linker 1 is a C₂₋₁₂-alkylene bridge, an ethylene glycol bridge containing 1 to 10 ethylene glycol units or a glycerol based bridge of the formula

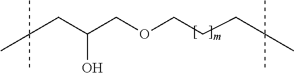

wherein m is an integer of 1 to 6.

In a preferred embodiment of the radiolabeled oligonucleotide of the formula Id, R^{1*} is methyl or ethyl, more preferably ethyl.

In another preferred embodiment of the radiolabeled oligonucleotide of the formula Id, X² is S.

In another preferred embodiment of the radiolabeled oligonucleotide of the formula Ic the linker 1 is a C₂₋₁₂-alkylene bridge, preferably a C₆-alkylene bridge.

Even more preferred is the radiolabeled oligonucleotide of the formula Id, wherein R^{1*} is methyl or ethyl, preferably ethyl; X² is S and the linker 1 is a C₆-alkylene bridge.

The radiolabeled oligonucleotide of the formula Id can be illustrated with the following compounds.
Prop-5′-Am-C6*G*C*a*t*t*g*g*t*a*t*T*C*A
G*A*G*t*t*a*c*t*t*g*c*c*a*A*C*T*-Am-GBB-Prop
G*C*a*t*t*g*g*t*a*t*T*C*A*-Am-GBB-Prop
wherein Am-C6 means a C6 (hexylene) amino linker; Am-GBB means a glycerol based bridge (m=1) amino linker, Prop is a ³H labeled propionyl; * stands for phosphorthioate bridges; A,C,G,T are LNA nucleoside monomers and a,t,c,g are DNA nucleoside monomers.

Most preferred embodiments are the radiolabeled oligonucleotide of the formula Ib and Ic.

The radiolabeled oligonucleotide of the formula Ib and Ic can be illustrated with the following compounds.
5′-GN2-C6-caG*A*G*t*t*a*c*t*t*g*c*c*a*A*C*T*-C6SH-NEM
G*C*a*t*t*g*g*t*a*t*T*C*A*-C6SH-NEM
G*C*a*t*t*g*g*t*a*t*T*C*A*-C6SH-NMM
G*A*G*t*t*a*c*t*t*g*c*c*a*A*C*T*-C6SH-NEM
G*A*G*t*t*a*c*t*t*g*c*c*a*A*C*T*-C6SH-NMM
5′-NEM-SH-C6*T*T*A*c*A*c*t*t*a*a*t*t*a*t*a*c*t*T*C*C
wherein C6SH means a C6 (hexylene) thiol linker; NEM is a ³H labeled N-ethylmaleimide; NMM is a ³H labeled N-methylmaleimide; * stands for phosphorthioate bridges; A,C,G,T are LNA nucleoside monomers and a,t,c,g are DNA nucleoside monomers.

The radiolabeled oligonucleotides of the present invention have a specific activity of 37 GBq/mmol (1 Ci/mmol) to 3.7 TBq/mmol (100 Ci/mmol), preferably of 111 GBq/mmol (3 Ci/mmol) to 1.85 TBq/mmol (50 Ci/mmol), more preferably of 185 GBq/mmol (5 Ci/mmol) to 740 GBq/mmol (20 Ci/mmol).

The invention also comprises a process for the preparation of a radiolabeled oligonucleotide of the formula I.

For those radiolabeled oligonucleotides of the formula I wherein Q stands for the residue of the formula 2a the process comprises conjugating an amine of formula III

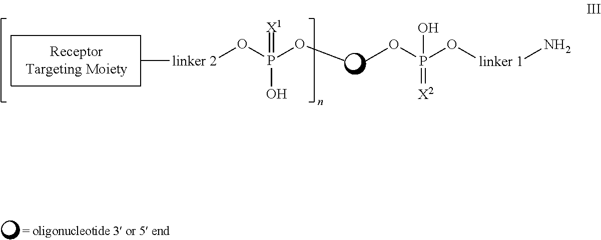

wherein,
X¹ and X² independently of each other are S or O;
linker 1 is a C₂₋₁₂-alkylene bridge, an ethylene glycol bridge containing 1 to 10 ethylene glycol units or a glycerol based bridge of the formula

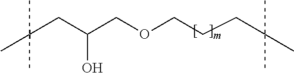

wherein m is an integer of 1 to 6;
linker 2 is an optionally amino group protected amino C₂₋₁₂-alkylene bridge, an amino ethylene glycol bridge containing 1 to 10 ethylene glycol units;
and the receptor targeting moiety is a moiety which adds additional functionality to the oligonucleotide;
with a radiolabeled succinimidyl compound of formula IV

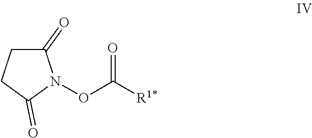

wherein R^{1*} is as above.

Radiolabeled succinimidyl derivatives are commercially available. The ³H labeled succinimidyl compound of formula IV with R^{1*} ethyl (N-succinimidyl propionate; NSP) can for instance be obtained from Pharmaron, Cardiff, UK.

The conjugation reaction can be performed in the presence of an organic base and an organic solvent or in an aqueous buffered system at a reaction temperature of 0° C. to 50° C.

Suitable organic bases are tertiary amines such as N,N-diisopropylethylamine (Hünig's base).

Suitable aqueous buffers such as phosphate-buffered saline in pH range of 6 to 9.

Suitable solvents are polar aprotic solvents such as N,N-dimethylformamide or dimethylsulfoxide.

The reaction mixture containing the resulting radiolabeled oligonucleotide can be freed from the solvent and the crude can be dissolved in a suitable aqueous buffer solution for further purification.

The purification essentially comprises the steps chromatography, concentration and isolation applying techniques well known to the skilled in then art.

The chromatography is a preparatory HPLC typically with a C-18 reversed-phase column using aqueous and organic solvents as mobile phases.

The concentration of the fractions obtained from the chromatography can take place via a tangential flow filtration, particularly a diafiltration over a suitable membrane.

Finally, the isolation of the radiolabeled oligonucleotide from the eluent can typically take place by lyophilization.

For those radiolabeled oligonucleotides of the formula I wherein Q stands for the residue of the formula 2b the process comprises conjugating a thiol of formula V

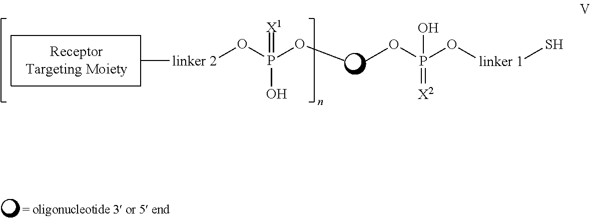

wherein,
X¹ and X² independently of each other are S or O;
linker 1 is a C₂₋₁₂-alkylene bridge, an ethylene glycol bridge containing 1 to 10 ethylene glycol units or a glycerol based bridge of the formula

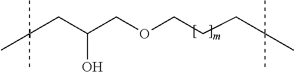

wherein m is an integer of 1 to 6;
linker 2 is an optionally amino group protected amino C₂₋₁₂-alkylene bridge, an amino ethylene glycol bridge containing 1 to 10 ethylene glycol units;
and the receptor targeting moiety is a moiety which adds additional functionality to the oligonucleotide;
with a radiolabeled maleimide compound of formula VI

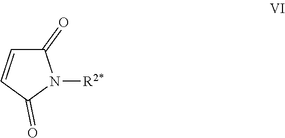

wherein R^{2*} is as above.

Radiolabeled maleimide derivatives are commercially available. The ³H labeled maleimide with R^{2*} methyl (Supplier 1) or ethyl (Supplier 2) can for instance be obtained from RC Tritec, Teufen, CH (Supplier 1), Pharmaron, Cardiff, UK (Supplier 2)

The conjugation reaction can be performed in the presence of an organic solvent at a reaction temperature of 0° C. to 50° C.

Suitable solvents are polar aprotic solvents such as N,N-dimethylformamide, dimethylsulfoxide or aqueous buffered systems.

The reaction mixture containing the resulting radiolabeled oligonucleotide can be freed form the solvent and the crude can be dissolved in a suitable aqueous buffer solution for further purification.

The purification essentially comprises the steps concentration and isolation applying techniques well known to the skilled in then art.

The concentration can take place via a tangential flow filtration, particularly a diafiltration of the aqueous solution over a suitable membrane.

The invention further comprises the use of the radiolabeled oligonucleotide for the determination of the biodistribution and pharmacokinetics of the oligonucleotide in the tissue or body fluid. In addition, tritium labeled oligonucleotides can be applied in bioscience, including quantitative whole body autoradiography (QWBA), target binding, and transporter efflux and uptake studies.

The invention also comprises a method for the determination of the biodistribution and pharmacokinetics of an oligonucleotide in the tissue or body fluid comprising
a) administering an effective amount of radiolabeled oligonucleotide of anyone of claims 1 to 11 to the tissue or the body fluid to be examined and
b) measuring the biodistribution and the pharmacokinetics of the radiolabeled oligonucleotide of anyone of claims 1 to 11 in the tissue or body fluid and optionally
c) imaging the radiolabeled oligonucleotide of anyone of claims 1 to 11 in the tissue or the body fluid to be examined by autoradiography.

The invention further comprises the oligonucleotide of the formula X

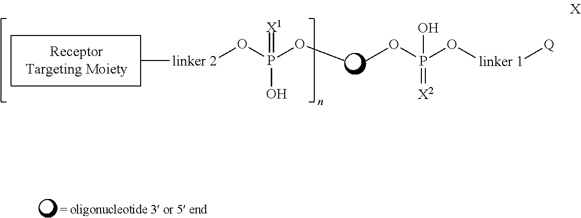

wherein,
n is 0 or 1;
X¹ and X² independently of each other are S or O;
linker 1 is a C₂₋₁₂-alkylene bridge, an ethylene glycol bridge containing 1 to 10 ethylene glycol units or a glycerol based bridge of the formula

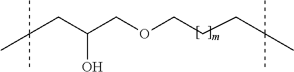

wherein m is an integer of 1 to 6;
linker 2 is an optionally amino group protected amino C₂₋₁₂-alkylene bridge, an amino ethylene glycol bridge containing 1 to 10 ethylene glycol units;
Q stands for a residue of the formula 2a′ or 2b′

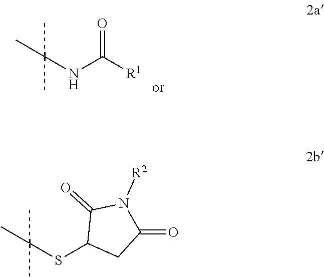

wherein R¹ and R² are C₁₋₆-alkyl groups; and
the receptor targeting moiety is a moiety which adds additional functionality to the oligonucleotide.

The preferred embodiments described for the radiolabeled oligonucleotides of formula I likewise applies for the oligonucleotides of formula X.

Accordingly, R¹ and R² stand for a C₁₋₄-alkyl group, preferably for a methyl or ethyl group more preferably for an ethyl group.

The preferred embodiments described for the radiolabeled oligonucleotides of formula Ib, Ic and Id likewise apply for the oligonucleotides of formula Xb

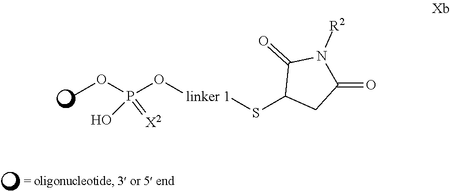

wherein R², X² and linker 1 are as above;
for the oligonucleotide of the formula Xc

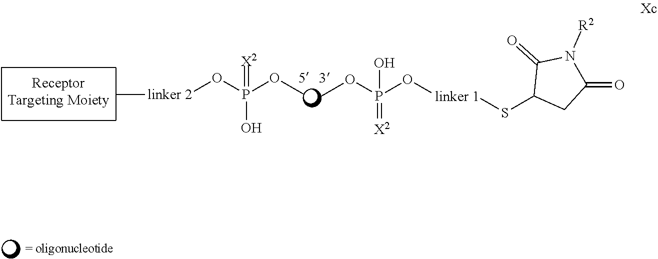

wherein R², X¹ and X², linker 1 and linker 2 are as above;
for the oligonucleotide of the formulae Xd

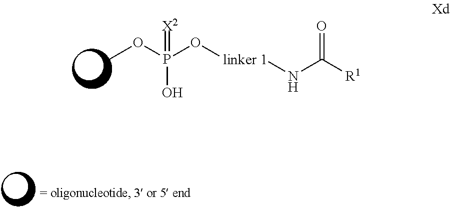

wherein R¹, X² and linker I are as above and the
the receptor targeting moiety which is a non-nucleotide moiety, preferably a asialglycoprotein receptor targeting moiety, more preferably a GalNAc moiety of formula VII

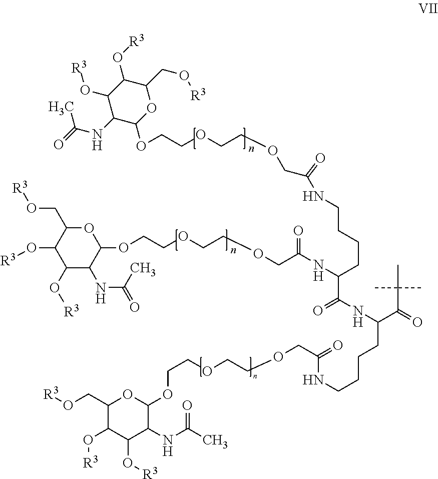

wherein R³ is hydrogen or a hydroxy protecting group and n is an integer from 0 to 10, preferably from 0 to 5, more preferably from 1 to 3, but most preferred is 2, corresponding salts, enantiomers and/or a stereoisomer thereof.

The compounds disclosed herein have the following nucleobase sequence.

(Oligo 1,3,5)
SEQ ID NO 1:
gcattggtattca
(Oligo 2,6)
SEQ ID NO 2:
gagttacttgccaact
(Oligo 4)
SEQ ID NO 3:
cagagttacttgccaact
(Oligo 7)
SEQ ID NO 4:
ttacacttaattatacttcc

### EXAMPLES

### Abbreviations

AcOH acetic acid
Bq Becquerel
Ci curries
Da Dalton
DI deionized
DIPEA N,N-diisopropylethylamine (Hünig's base)
DMAP 4-(dimethylamino)-pyridine
DMF N,N-dimethylformamide
DMSO dimethylsulfoxide
EtOH ethanol
GBB glycerol based bridge
h hours
HPLC High-performance liquid chromatography
i iso
LC-MS/MS Liquid chromatography coupled to tandem mass spectrometry
LNA Locked nucleic acid
LSC Liquid scintillation counting
MeOH methanol
min minutes
mM, nM Millimolar, Nanomolar
mL Mililitre
μL Microloliter
MS mass spectrometry
MW molecular weight
MWCO molecular weight cut off
n normal
NEM N-ethyl maleimide
ng Nanogram
nm Nanometer
NMM N-methyl maleimide
NSP N-succinimidyl propionate
p para
PBS phosphate-buffered saline
PCR Polymerase chain reaction
PD Pharmacodynamic
Prop propionate
QC Quality Control (sample)
QWBA quantitative whole body autoradiography
rpm round per minutes
rt room temperature
SRM Selected reaction monitoring
t tertiary
TEA triethylamine
UPLC Ultra-performance liquid chromatography
v Volume

### General Methods

All oligonucleotides, which were use as starting materials, were synthesized from Roche Pharma research and early development. Tritium labeled N—[³H]ethyl maleimide (specific activity: 2 TBq/mmol=55 Ci/mmol) was obtained from Pharmaron (Cardiff, Wales, UK) as solution in pentane. Tritium labeled N—[³H]succinimidyl propionate (specific activity: 3.8 TBq/mmol=103 Ci/mmol) was obtained from RC Tritec (Teufen, CH) as solution in toluene. Liquid scintillation counting for tritium compounds was accomplished using a HIDEX 300 SL and ULTIMATE GOLD cocktail (PerkinElmer Inc., Waltham, MA, USA). Reaction monitoring and purity for Oligos 1-3 were determined by HPLC Agilent 1210 at 260 nM wavelength, Waters XBridge RP18, 4.6×150 mm, 3.5 μm column at 60° C. ([A]=water/methanol/hexafluoro i-propanol/TEA: 950/25/21/2.3 mL; [B]=water/methanol/hexafluoro i-propanol/TEA: 175/800/21/2.3 mL) at flow 1.0 mL/min with the following gradient: 10% [B] to 60% [B] in 12 min. Oligos 4-6 were determined by UPLC Agilent 1290 at 260 nm wavelength, ACQUITY UPLC Oligonucleotide BEH C18, 2.1×50 mm, 1.7 μm column at 80° C. with same eluents and the following gradient: 10% [B] to 40% in 6 min. Oligo 7 was analyzed with same condition like Oligos 4-6 accept the following gradient: 10% [B] to 30% in 6 min. Mass spectrometry was perfomed by Waters Acquity UPLC H-class System equiped with Single Quadruple (SQ) and ESI Mass Detector Radiochemical purity was measured using the β-radioactivity HPLC detector RAMONA Quattro with internal solid scintillator (Raytest, Straubenhardt, Germany). Preparative HPLC for Oligos 1-3 were performed by Gilson PLC 2050 with XBridge C18 column, 5 μm, 10 mm×250 mm and using water (950 mL)/methanol (25 mL)/TEA (2.3 mL)/hexafluoro i-propanol (21 mL) as mobile phase [A] and water (175 ml)/methanol (800 mL)/TEA (2.3 mL)/hexafluoro i-propanol (21 mL) as mobile phase [B] as gradient with 10% [B] to 60% [B] in 15 minutes. Concentration was determined by Eppendorf BioSprectrometer basic at 260 nm wavelength and the corresponding calculated molar extinction coefficient.

### Example 1 (Non-Radioactive Conjugation on Amine Linker)

### a) Oligonucleotides Used in the Examples

5-Am-C6*G*C*a*t*t*g*g*t*a*t*T*C*A; MW: 4520.7 g/mol; (Oligo 1)
G*A*G*t*t*a*c*t*t*g*c*c*a*A*C*T*-Am-GBB; MW: 5506.5 g/mol, (Oligo 2)
G*C*a*t*t*g*g*t*a*t*T*C*A*-Am-GBB: MW: 4553.6 g/mol, (Oligo 3)

### b) General Mode of Reaction

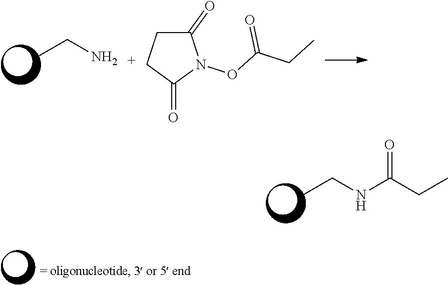

### c) General Procedure

To 1 equivalent of oligo nucleotide, containing an amine linker on 5′ or 3′ end in DMF (volume factor: 125 mL/g) and 40 equivalent Hünig's base was added 1.2 equivalent N-succinimidyl propionate (NSP) to give a colorless suspension. The mixture stirred over night at room temperature to become a clear and colorless solution. The solvent was removed under high vacuum and the residue dissolved in PBS. Crude mixture was purified by preparative HPLC. The desired fractions were transferred into an Amicon® Pro purification system (MWCO: 3.000 Da) and centrifuged at 4000 rpm. DI water was added and the process was repeated 4 times more to complete an exchange from HPLC eluent to water. The resulting aqueous solution was lyophilized to isolate the oligonucleotide as a colorless powder with a yield in range of 47%-74% and 96%-99% purity.

In accordance with the general procedure (1.c.) the oligonucleotides Oligo 1-3 have been conjugated.

### Example 1.d. (Conjugate 1)

5-Am-C6*G*C*a*t*t*g*g*t*a*t*T*C*A; Yield: 47%, purity: 99%, MS (m/z): 4577.0 [M-(H)]⁻

### Example 1.e. (Conjugate 2)

G*A*G*t*t*a*c*t*t*g*c*c*a*A*C*T*-Am-GBB-Prop; Yield: 74%, purity: 95%, MS (m/z): 5561.6 [M-(H)]⁻

### Example 1.f. (Conjugate 3)

G*C*a*t*t*g*g*t*a*t*T*C*A*-Am-GBB-Prop; Yield: 60%, purity: 96 % , MS (m/z): 4662.3 [M-(H)]⁻

### Example 2 (Non-Radioactive Conjugation on Thiol Linker)

### a) Oligonucleotides Used in the Examples

5′-GN2-C6-caG*A*G*t*t*a*c*t*t*g*c*c*a*A*C*T*-C6SH; MW: 7709.5 g/mol; (Oligo 4)
G*C*a*t*t*g*g*t*a*t*T*C*A*-C6SH; MW: 4537.6 g/mol; (Oligo 5)
G*A*G*t*t*a*c*t*t*g*c*c*a*A*C*T*-C6SH; MW: 5491.5 g/mol, (Oligo 6)
5′-SH-C6*T*T*A*c*A*c*t*t*a*a*t*t*a*t*a*c*t*T*C*C; MW: 6742.3 g/mol, (Oligo 7)

### b) General Mode of Reaction

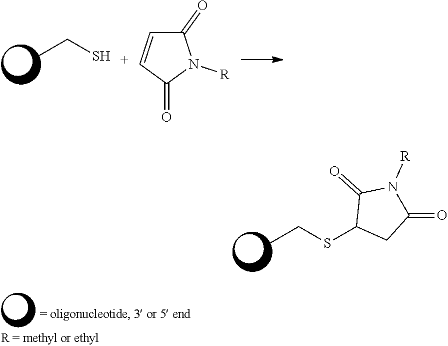

### c) General Procedure

1 equivalent of oligonucleotide with 5′ or 3′ end sulfhydryl linker was dissolved in PBS (volume factor: 250 mL/g). 1.5 equivalent of N-alkylated maleimide (methyl or ethyl), dissolved in DMSO (volume factor: 1500 mL/g), was added to the aqueous solution and stirred at room temperature for 1 h. UPLC analysis showed a complete addition of maleimide to oligo nucleotide. To exchange the buffer to water, the reaction mixture was transferred into an Amicon® Pro purification system (MWCO: 3.000 Da) and centrifuged at 4000 rpm. DI water was added and the process was repeated 4 times more to complete the exchange. The resulting aqueous solution was lyophilized to isolate the oligonucleotide as a colorless powder with a yield in range of 69%-81% and 96%-99% purity.

In accordance with the general procedure (2.c.) the oligonucleotides Oligo 4-7) have been conjugated.

### Example 2.d. (Conjugate 4)

5′-GN2-C6-caG*A*G*t*t*a*c*t*t*g*c*c*a*A*C*T*-C6SH-NEM; Yield: 73%, purity: 99%, MS (m/z): 7833.4 [M-(H)]⁻

### Example 2.e. (Conjugate 5)

G*C*a*t*t*g*g*t*a*t*T*C*A *-C6SH-NEM, Yield: 73%, purity: 99%, MS (m/z): 4662.3 [M-(H)]⁻

### Example 2.f. (Conjugate 6)

G*C*a*t*t*g*g*t*a*t*T*C*A*-C6SH-NMM; Yield: 81%, purity: 99%, MS (m/z): 4648.2 [M-(H)]⁻

### Example 2.g. (Conjugate 7)

G*A*G*t*t*a*c*t*t*g*c*c*a*A*C*T*-C6SH-NEM; Yield: 73%, purity: 96 % , MS (m/z): 5616.2 [M-(H)]⁻. ¹H NMR (600 MHz, D₂O) δ ppm 4.11 (br s, 2 H), 4.05-4.17 (m, 1 H), 3.75 (br s, 2 H), 3.43-3.52 (m, 1 H), 2.80-2.91 (m, 1 H), 2.74-2.91 (m, 2 H), 1.71-1.83 (m, 2 H), 1.61-1.77 (m, 2 H), 1.44-1.59 (m, 2 H), 1.43-1.56 (m, 2 H), 1.34 (br s, 3 H)

NMR data limited to linker and NEM conjugated label.

### Example 2.h. (Conjugate 8)

G*A*G*t*t*a*c*t*t*g*c*c*a*A*C*T*-C6SH-NMM; Yield: 69%, purity: 97%, MS (m/z): 5602.2 [M-(H)]⁻

### Example 2.i. (Conjugate 9)

5′-NEM-SH-C6*T*T*A*c*A*c*t*t*a*a*t*t*a*t*a*c*t*T*C*C; Yield: 97%, purity: 99%, MS (m/z): 6868.7 [M-(H)]⁻

### Example 3 (Radioactive Conjugation Oligonucleotides)

### Example 3.a. ([³H]-Compound 1 Based on Conjugate 2)

### G*A*G*t*t*a*c*t*t*g*c*c*a*A*C*T*-Am-GBB-[³H]-Prop

370 MBq (10 mCi) of N—[³H]succinimidyl propionate (17.3 μg, 0.079 μmol) with a specific activity of 3.811 GBq/mmol (103 Ci/mmol) and dissolved in 2 mL toluene was diluted with 22.8 μg of the corresponding non-radioactive N-succinimidyl propionate to achieve a total amount of 40.1 μg (0.234 μmol) with a specific activity of 1.554 TBq/mmol (42 Ci/mmol). The solvent was removed by evaporation and the solid residue was dissolved in 100 μl DMF. 0.98 mg (0.167 μmol) of Olio 2, dissolved in 250 μL DMF and 1.3 μL (0.97 μmol) DIPEA, was dropped to the [³H]NSP solution and stirred over night at room temperature. UPLC showed a conversion of 40% to the desired product. The reaction solution was filled into an Amicon® Pro purification system (MWCO: 3.000 Da) and centrifuged at 4000 rpm to change the solvent to water/methanol/hexafluoro i-propanol/TEA: 950/25/21/2.3 for preparative HPLC sample preparation. After prep-HPLC, the corresponding fraction was deluted with PBS and transferred into an Amicon® Pro purification system (MWCO: 3.000 Da) and centrifuged at 4000 rpm. PBS was added and the process was repeated 4 times more to achieve a chemical purity of 99%. Volume: 0.55 mL, concentration: 0.32 mg/mL, amount: 0.19 mg (yield: 19.5%), activity: 51.8 MBq (1.4 mCi), specific activity: 262.7 MBq/mg (7.1 mCi/mg) which is equal to 1.554 TBq/mmol (42 Ci/mmol).

### Example 3.b. ([³H]-Compound 2 Based on Conjugate 4)

### 5′-GN2-C6-caG*A*G*t*t*a*c*t*t*g*c*c*a*A*C*T*-C6SH-[³H]-NEM

370 MBq (10 mCi) of N—[³H]ethyl maleimide (20.5 μg, 0.159 μmol) in 4 mL pentane was concentrated on a silica gel pre-packed column and eluted with 2×0.5 mL DMSO. A solution of Oligo 4 (1.02 mg, 0.132 μmol) in 1 mL PBS was added and stirred 1 h at room temperature. UPLC analysis showed 20% of the desired product. Non-radioactive NEM (166 μg, 1.32 μmol) was added and stirred at room temperature for 1 h. HPLC showed a complete addition to the desired product. The reaction solution was transferred into a 5 mL Float-A-Lyzer® tube (MWCO: 500-1000 Da) and dialyzed against PBS pH 7.1 at room temperature. Buffer was changed 4 times after 45 minutes and stored overnight in the fridge. UPLC showed a radio chemical purity of 93%. Volume: 2.9 mL, concentration: 0.33 mg/mL, amount: 0.95 mg (yield: 92%), activity: 33.7 MBq (0.91 mCi), specific activity: 35.5 MBq/mg (953 μCi/mg) which is equal to 0.3 TBq/mmol (7.9 Ci/mmol).

### Example 3.c. ([³H]-Compound 3 Based on Conjugate 7)

### G*A*G*t*t*a*c*t*t*g*c*c*a*A*C*T*-C6SH-[³H]-NEM

1.1 GBq (30 mCi) of N—[³H]ethyl maleimide (61.5 μg, 0.477 μmol) in 12 mL pentane was concentrated on a silica gel pre-packed column and eluted with 2×0.5 mL DMSO. A solution of Oligo 6 (2.20 mg, 0.401 μmol) in 1 mL PBS was added and stirred 1 h at room temperature. UPLC analysis showed 40% of the desired product. Non-radioactive NEM (502 μg, 4.01 μmol) was added and stirred at room temperature for 1 h. HPLC showed a complete addition to the desired product. The reaction solution was transferred into a 5 mL Float-A-Lyzer® tube (MWCO: 500-1000 Da) and dialyzed against PBS pH 7.1 at room temperature. Buffer was changed 4 times after 45 minutes and stored overnight in the fridge. UPLC showed a high polar radio impurity. The solution was filled into an Amicon® Pro purification system (MWCO: 3.000 Da) and centrifuged at 4000 rpm. PBS was added and the process was repeated 4 times more to achieve a chemical purity of 99%. Volume: 1.0 mL, concentration: 1.58 mg/mL, amount: 1.58 mg (yield: 70%), activity: 163 MBq (4.4 mCi), specific activity: 104 MBq/mg (2.8 mCi/mg) which is equal to 614 MBq/mmol (16.6 Ci/mmol).

### Example 3.d. ([³H]-Compound 4 Based on Conjugate 9)

### 5′-[³H]-NEM-SH-C6*T*T*A*c*A*c*t*t*a*a*t*t*a*t*a*c*t*T*C*C

370 MBq (10 mCi) of N—[³H]ethyl maleimide (20.5 μg, 0.159 μmol) in 4 mL pentane was concentrated on a silica gel pre-packed column and eluted with 2×0.5 mL DMSO, dropped into a solution of Oligo 7 (1.13 mg, 0.168 μmol) in 0.5 mL PBS and let it stir for 1.5 h at rt. UPLC showed 45% desired product and 55% starting material. Non-radioactive NEM (210 μg, 1.68 μmol) was added and stirred at room temperature for 1 h. HPLC showed a complete addition to the desired product. The reaction solution was transferred into an Amicon® Pro purification system (MWCO: 3.000 Da) and centrifuged at 4000 rpm. PBS was added and the process was repeated 4 times more to achieve a chemical purity of 99%. Volume: 1.0 mL, concentration: 1.80 mg/mL, amount: 1.07 mg (yield: 93%), activity: 71 MBq (1.91 mCi), specific activity: 67 MBq/mg (1.8 mCi/mg) which is equal to 481 MBq/mmol (13.0 Ci/mmol).

### Tissue Exposure Study of Unlabeled and Tritium Labeled LNA—a Feasibility Study

The studies have been performed with the following compounds:
5′-GN2-C6-ca G*A*G*t*t*a*c*t*t*g*c*c*a*A*C*T*-3′ (GalNAc LNA study compound A)
5′-G*A*G*t*t*a*c*t*t*g*c*c*a*A*C*T*-3′ (LNA study compound A)
5′-GN2-C6-caG*A*G*t*t*a*c*t*t*g*c*c*a*A*C*T*-C6SH-[³H]-NEM (=Example 3.b)
5′-G*A*G*t*t*a*c*t*t*g*c*c*a*A*C*T*-C6SH-[³H]-NEM (=Example 3.c)

A single dose PK experiment with Example 3.b ([³H]-compound 2 based on conjugate 4) and Example 3.c ([³H]-compound 3 based on conjugate 7) at 1 mg/kg was done. LNAs were analyzed in liver 24, 72 and 336 h after dosing. The study will confirm the feasibility of oligonucleotides with radioactive conjugation.

### Experimental Part for In Vivo Study

### Preparation of Stock Solution, Calibration Standards and Quality Checks

Serial dilutions were made from stock solution (approx. 1 mg/mL in PCR grade water, the exact concentration of the stock solution will be quantified with the spectro-photometric device Nanodrop (Thermo Scientific) based on the extinction coefficient at 260 nm) to generate working solutions in water from approx. 100 ng/mL up to approx. 250000 ng/mL.

These working solutions were used to spike plasma following this procedure: 1 μL working solution was added to 49 μL plasma in order to create calibration samples, and quality control samples at 4 concentration levels in plasma.

### Extraction Method

Calibration standards and quality control samples (freshly prepared in plasma, 50 μL) were treated for protein denaturation with 150 μL of 4 M guanidine thiocyanate after addition of the internal standard. After vigorously mixing (20 min at 1600 rpm), 200 μL of a water/hexafluoroisopropanol/diisopropylethylamine solution (100:4:0.2, v/v/v) were added, followed by mixing (15 min at 1500 rpm). Then a clean-up step was performed by means of solid-phase-extraction cartridges (Waters, OASIS HLB 5 mg, 30 μm) after elution and evaporation to dryness (30-45 min at +40° C.) the samples were reconstituted in 200 μL of mobile phase (water/methanol/hexafluoroisopropanol/diisopropylethylamine (95/5/1/0.2, v/v/v/v)). After vortex mixing (10 min at 1500 rpm), an aliquot (20 μL) was injected into a LC-MS/MS system (50 μL loop).

### Description of LC-MS/MS Method

A Shimadzu 30ADXR pump was used, equipped with a Waters Acquity C18 column (50×2.1 mm) at 60° C. The analytes and internal standard were separated from matrix interferences using gradient elution from water/methanol/hexafluoroisopropanol/diisopropyletylamine (95/5/1/0.2, v/v/v/v) to water/methanol/hexafluoroisopropanol/diisopropyletylamine (10/90/1/0.2, v/v/v/v) within 4.0 min at a flow rate of 0.4 mL/min.

Mass spectrometric detection was carried out on an AB-Sciex Triple Quad 6500⁺ mass spectrometer using SRM in the negative ion mode.

### Liquid Scintillation Counting

A Packard Tri-carb 3100TR was used for LSC analysis.

### Detailed Description of the Figures

In FIG. 1 the liver concentration of a GalNAc LNA study compound A (dotted line) and the LNA study compound A without GalNAc (continuous line) have been analyzed by LC-MS/MS. The GalNAc labeled LNA shows as expected a high initial uptake in the liver plasma and a normal decrease over the time. Likewise shows the naked, i.e. not GalNAc containing LNA, a lower level of uptake.

In FIG. 2 the liver concentration of the tritium labeled compounds of Example 3.b (dotted line) and Example 3.c (continuous line) have been analyzed by LSC. This figure shows, that the radiolabeled GalNAc compound, despite of the maleimide conjugation, has an equivalent liver uptake as a therapeutic GalNAc LNA (FIG. 1).

PD effects are comparable for the unlabeled and radio labeled oligonucleotide. LNA concentration measurements in the liver of the radioactivity by LSC is similar to the therapeutic LNAs, determined by LC-MS/MS.

FIG. 2 impressively illustrates the high specificity of the radiolabeled oligonucleotide compounds of the present invention.

1-24. (canceled)

25. A method of preparing a radiolabeled oligonucleotide of formula I: 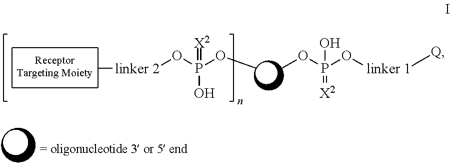
wherein
n is 0 or 1;
X¹ and X² independently of each other are S or O;
linker 1 is a C₂₋₁₂-alkylene bridge, an ethylene glycol bridge containing 1 to 10 ethylene glycol units or a glycerol-based bridge of the formula: 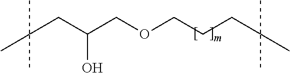
wherein m is an integer of 1 to 6;
linker 2 is an optionally amino group protected amino C₂₋₁₂-alkylene bridge or an amino ethylene glycol bridge containing 1 to 10 ethylene glycol units;
the receptor targeting moiety is a GalNAc moiety;
Q represents a residue of formula 2a: 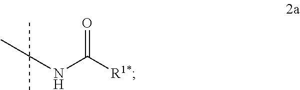
and
R^{1*} is radiolabeled C₁₋₆-alkyl group,
comprising conjugating an amine of formula II: 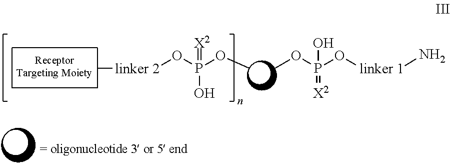
with a radiolabeled succinimide compound of formula IV:

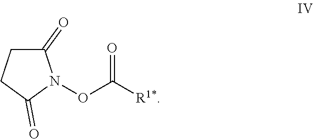

26. A method of preparing a radiolabeled oligonucleotide of formula I: 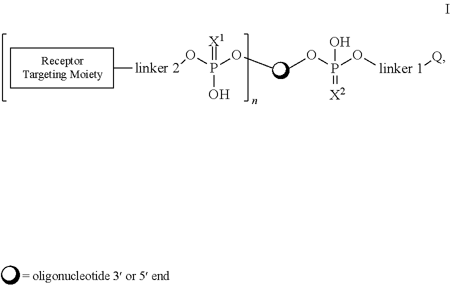
wherein
n is 0 or 1;
X¹ and X² independently of each other are S or O;
linker 1 is a C₂₋₁₂-alkylene bridge, an ethylene glycol bridge containing 1 to 10 ethylene glycol units, or a glycerol-based bridge of the formula: 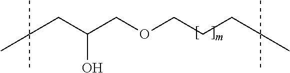
wherein m is an integer of 1 to 6;
linker 2 is an optionally amino group protected amino C₂₋₁₂-alkylene bridge or an amino ethylene glycol bridge containing 1 to 10 ethylene glycol units;
the receptor targeting moiety is a GalNAc moiety;
Q represents a residue of formula 2b: 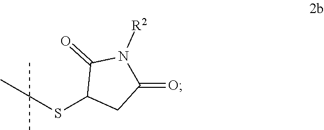
and
R^{2*} is a radiolabeled C₁₋₆-alkyl group,
comprising conjugating a thiol of formula V: 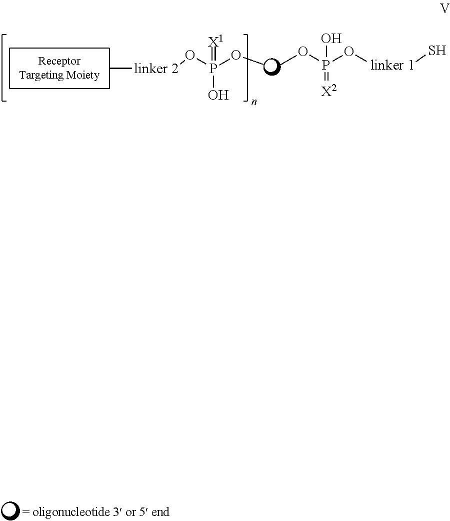
with a radiolabeled maleinimide compound of formula VI:

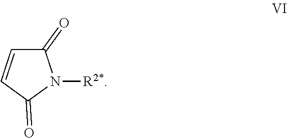

27. The process of claim 25, wherein the receptor targeting moiety is a GalNAc moiety of formula VII: 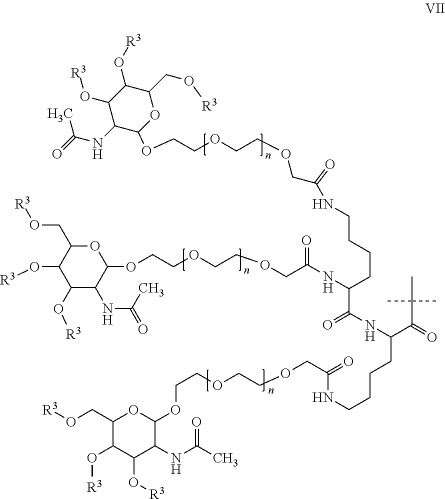
or a salt, enantiomer, or stereoisomer thereof, wherein,
R³ is hydrogen or a hydroxy protecting group; and
n is an integer from 0 to 10.

28. The process of claim 26, wherein the receptor targeting moiety is a GalNAc moiety of formula VII: 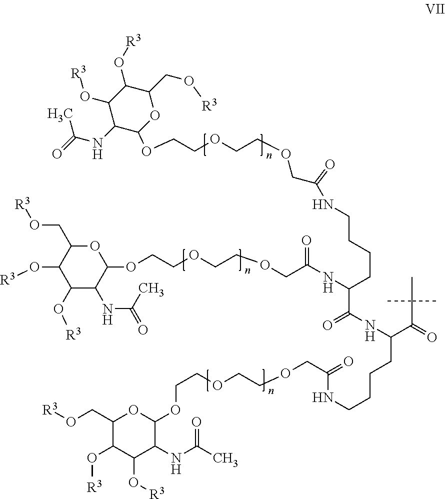
or a salt, enantiomer, or stereoisomer thereof, wherein,
R³ is hydrogen or a hydroxy protecting group; and
n is an integer from 0 to 10.

29. The process of claim 25, wherein R^{1*} is a ³H-radiolabeled C₁₋₆-alkyl group.

30. The process of claim 26, wherein R^{2*} is a ³H-radiolabeled C₁₋₆-alkyl group.

31. The process of claim 25, wherein the oligonucleotide comprises a contiguous nucleotide sequence of 7 to 30 nucleotides consisting of optionally modified DNA, RNA or LNA nucleoside monomers or combinations thereof.

32. The process of claim 26, wherein the oligonucleotide comprises a contiguous nucleotide sequence of 7 to 30 nucleotides consisting of optionally modified DNA, RNA or LNA nucleoside monomers or combinations thereof.

33. The process of claim 26, wherein the radiolabeled oligonucleotide of formula I is a compound of formula Ic 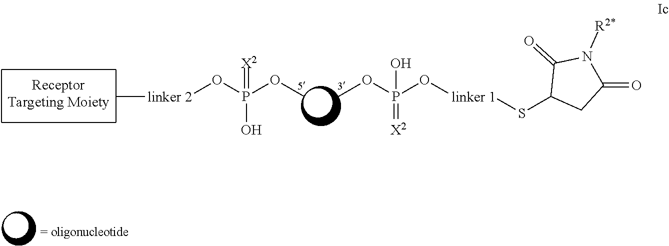
wherein R^{2*}, X¹ and X², linker 1 and linker 2 are the same as for formula I.

34. The process of claim 25, wherein the radiolabeled oligonucleotide of formula I has a specific activity of 37 GBq/mmol (1 Ci/mmol) to 3.7 TBq/mmol (100 Ci/mmol).

35. The process of claim 26, wherein the radiolabeled oligonucleotide of formula I has a specific activity of 37 GBq/mmol (1 Ci/mmol) to 3.7 TBq/mmol (100 Ci/mmol).


SEQUENCE LISTING (4 sequences)

SEQ ID NO: 1 - DNA, 13 bp, synthetic construct
  misc_feature 1..13: note = Oligonucleotide Sequence
  source 1..13: mol_type = other DNA
gcattggtattca

SEQ ID NO: 2 - DNA, 16 bp, synthetic construct
  misc_feature 1..16: note = Oligonucleotide Sequence
  source 1..16: mol_type = other DNA
gagttacttgccaact

SEQ ID NO: 3 - DNA, 18 bp, synthetic construct
  misc_feature 1..18: note = Oligonucleotide Sequence
  source 1..18: mol_type = other DNA
cagagttacttgccaact

SEQ ID NO: 4 - DNA, 20 bp, synthetic construct
  misc_feature 1..20: note = Oligonucleotide Sequence
  source 1..20: mol_type = other DNA
ttacacttaattatacttcc